(19) United States
(12) Patent Application Publication
(10) Pub. No.: US 2022/0259169 A1
(43) Pub. Date: Aug. 18, 2022

(54) MULTI-STAGE ESTERIFICATION PROCESS

(71) Applicant: DUPONT INDUSTRIAL BIOSCIENCES USA, LLC, Wilmington, DE (US)

(72) Inventors: Judy H. Cipot-Wechsler, Kingston (CA); Stuart B. Fergusson, Kingston (CA); Larin Godfroy, Napanee (CA); Ronnie Ozer, Arden, DE (US)

(21) Appl. No.: 17/737,408

(22) Filed: May 5, 2022

Related U.S. Application Data
(63) Continuation of application No. 17/188,914, filed on Mar. 1, 2021.
Application No. 17/188,914 is a continuation of application No. 16/863,481, filed on Apr. 30, 2020, now abandoned.
Application No. 16/863,481 is a continuation of application No. 15/580,395, filed on Dec. 7, 2017, now abandoned.
(60) Provisional application No. 62/196,795, filed on Jul. 24, 2015.

(51) Int. Cl. C07D 307/68

(52) U.S. Cl. CPC C07D 307/68

(57) ABSTRACT

A process for efficiently esterifying furan dicarboxylic acid, especially 2,5-furan dicarboxylic acid in the presence of an alcohol and, optionally, a catalyst is disclosed. The process provides high yields of the diesters of furan dicarboxylic acid and comprises a multi-stage esterification process wherein a portion of a gas phase is removed at each stage of the multi-stage process.

### CROSS-REFERENCE TO RELATED APPLICATION

This application is a Continuation of U.S. application Ser. No. 17/188914 filed on Mar. 1, 2021, which is a Continuation of U.S. application Ser. No. 16/863481 filed on Apr. 30, 2020, which is a Continuation of U.S. application Ser. No. 15/580395 filed on Dec. 7, 2017, which is a 371 of International Application No. PCT/US16/43309 filed Jul. 21, 2016, which claims benefit of priority of U.S. Provisional Application No. 62/196795 filed on Jul. 24, 2015, which is incorporated herein by reference in its entirety.

### FIELD OF THE DISCLOSURE

The present disclosure is directed towards a multi-stage esterification process that is especially useful for forming diesters of 2,5-furandicarboxylic acid.

### BACKGROUND OF THE DISCLOSURE

Derivatives of furan are known as potentially being useful in many industries, for example, pharmaceuticals, as fuel components, and as precursors for plastics. It has been disclosed that biomass materials can be used as a raw material to produce furan derivatives that can be useful as intermediates. For example, various sources of biomass can be hydrolyzed to produce pentose and hexose sugars which can then be further processed to form furfural and hydroxymethyl furfural (HMF). In order to be industrially useful, the furfural and HMF must be efficiently processed to the desired materials. One useful product is 2,5-furandicarboxylic acid, and esters thereof, which can be further processed to form various polymers, including polyesters. Polyesters comprising the furan dicarboxylates may have useful properties and could provide a replacement or partial replacement for polyesters derived from terephthalic acid. However, there is a continuing need for the efficient production of furandicarboxylic acid diesters that can be processed into a furan containing polyesters.

### SUMMARY OF THE DISCLOSURE

The present disclosure relates to a process comprising:
a) contacting 2,5-furan dicarboxylic acid (FDCA) with excess alcohol and, optionally, a catalyst in a first stage of a n-stage reactor at a temperature in the range of from 150° C. to 325° C. and a pressure in the range of from 0 bar to 140 bar to form a mixture comprising an ester of 2,5-furan dicarboxylic acid, the alcohol and water;
b) removing at least a portion of a gas phase from the first stage of the n-stage reactor;
c) allowing at least a portion of the mixture to move to the next sequential stage of the n-stage reactor system;
d) heating the mixture of step c) to a temperature in the range of from 50° C. to 325° C. and a pressure in the range of from 0 bar to 140 bar;
e) removing at least a portion of a gas phase from the mixture of step d);
f) repeating steps c), d) and e) until the last stage of the n-stage reactor system is reached; and
g) separating the ester of 2,5-furan dicarboxylic acid from the mixture,
wherein n is 2 to 10.

In other embodiments, the ester of 2,5-furan dicarboxylic acid of step g) is further purified by crystallization, distillation or a combination thereof.

In other embodiments, the gas phases removed in steps b) and e) comprise alcohol, water, the ester of 2,5-furan dicarboxylic acid or a combination thereof.

In other embodiments, the n-stage reactor system comprises one or more of a continuously stirred tank reactor, a plug flow reactor, a reaction column, a Scheibel column, a tank reactor or a combination thereof.

In other embodiments, the n-stage reactor system comprises continuously stirred tank reactors connected in series.

In other embodiments, the alcohol is methanol and the ester of 2,5-furan dicarboxylic acid is the dimethyl ester of 2,5-furan dicarboxylic acid (FDME).

In other embodiments the catalyst is cobalt (II) acetate, iron (II) chloride, iron (III) chloride, iron (II) sulfate, iron (III) sulfate, iron (II) nitrate, iron (III) nitrate, iron (II) oxide, iron (III) oxide, iron (II) sulfide, iron (III) sulfide, iron (II) acetate, iron (III) acetate, magnesium (II) acetate, magnesium (II) hydroxide, manganese (II) acetate, phosphoric acid, sulfuric acid, zinc (II) acetate, zinc stearate, a solid acid catalyst, a zeolite solid catalyst, or a combination thereof.

In other embodiments at least a portion of the alcohol is replaced with an alcohol source. In some embodiments, the alcohol source is an acetal, an orthoformate, an alkyl carbonate, a trialkyl borate, a cyclic ether comprising 3 or 4 atoms in the ring, or a combination thereof.

### DETAILED DESCRIPTION OF THE DISCLOSURE

The disclosures of all cited patent and non-patent literature are incorporated herein by reference in their entirety.

As used herein, the term “embodiment” or “disclosure” is not meant to be limiting, but applies generally to any of the embodiments defined in the claims or described herein. These terms are used interchangeably herein.

Unless otherwise disclosed, the terms “a” and “an” as used herein are intended to encompass one or more (i.e., at least one) of a referenced feature.

The features and advantages of the present disclosure will be more readily understood by those of ordinary skill in the art from reading the following detailed description. It is to be appreciated that certain features of the disclosure, which are, for clarity, described above and below in the context of separate embodiments, may also be provided in combination in a single element. Conversely, various features of the disclosure that are, for brevity, described in the context of a single embodiment, may also be provided separately or in any sub-combination. In addition, references to the singular may also include the plural (for example, “a” and “an” may refer to one or more) unless the context specifically states otherwise.

The use of numerical values in the various ranges specified in this application, unless expressly indicated otherwise, are stated as approximations as though the minimum and maximum values within the stated ranges were both proceeded by the word “about”. In this manner, slight variations above and below the stated ranges can be used to achieve substantially the same results as values within the ranges. Also, the disclosure of these ranges is intended as a continuous range including each and every value between the minimum and maximum values.

As used herein:

The term “solid acid catalyst” refers to any solid material containing Brönsted and/or Lewis acid sites, and which is substantially undissolved by the reaction medium under ambient conditions.

The phrase “n-stage reactor system” means 2 or more reaction vessels connected in series or it can also mean one or more reaction vessels where at least one of the reaction vessels is utilized more than once. The n-stage reactor system can comprise 2 to 10 reaction vessels connected in series.

The phrase “alcohol source” means a molecule which, in the presence of water and optionally an acid, forms an alcohol.

The acronym FDCA means 2,5-furan dicarboxylic acid.

The phrase “ester of FDCA” means a diester of 2,5-furan dicarboxylic acid.

The acronym FDME means the dimethyl ester of 2,5-furan dicarboxylic acid.

The acronym FDMME means the monomethyl ester of 2,5-furan dicarboxylic acid.

The acronym FFME means the methyl ester of 5-formylfuran-2-carboxylic acid.

The present disclosure relates to an efficient process for the formation of an ester of FDCA. The process comprises:
a) contacting 2,5-furan dicarboxylic acid with excess alcohol and, optionally, a catalyst in a first stage of a n-stage reactor at a temperature in the range of from 150° C. to 325° C. and a pressure in the range of from 0 bar to 140 bar to form a mixture comprising an ester of 2,5-furan dicarboxylic acid, the alcohol and water;
b) removing at least a portion of a gas phase from the first stage of the n-stage reactor;
c) allowing at least a portion of the mixture to move to the next sequential stage of the n-stage reactor system;
d) heating the mixture of step c) to a temperature in the range of from 50° C. to 325° C. and a pressure in the range of from 0 bar to 140 bar;
e) removing at least a portion of a gas phase from the mixture of step d);
f) repeating steps c), d) and e) until the last stage of the n-stage reactor system is reached; and
g) separating the ester of 2,5-furan dicarboxylic acid from the mixture,
wherein n is 2 to 10.

The process comprises a first step a) contacting FDCA with excess alcohol and, optionally a catalyst in a first stage of an n-stage reactor system at a temperature in the range of from 50° C. to 325° C. and a pressure in the range of from 0 bar to 140 bar. The n-stage reactor system can be any suitable reactors connected in series. For example, suitable reactors include for example, one or more of a continuously stirred tank reactor, a plug flow reactor, a reaction column, a Scheibel column, a tank reactor or a combination thereof. The n-stage reactor can also be a Scheibel column having between 2 and 10 plates. In some embodiments, the n-stage reactor system can be 2 to 10 continuously stirred tank reactors connected in series. In other embodiments, the n-stage reactor system can be 1 to 9 continuously stirred tank reactors connected in series with a plug flow reactor as the last reactor in the series. Each reactor should consist of an inlet for adding the raw materials, an outlet for discharging the contents of the reactor and an outlet for removing at least a portion of a gas phase from the reactor. In some embodiments, the n-stage reactor system can be run as a continuous process, for example, using a series of continuously stirred tank reactors, using one or more plug flow reactors or using a Scheibel column. In other embodiments, the n-stage reactor system can be run as a batch process, for example, using two or more tank reactors.

Each reactor can be operated at temperatures in the range of from 150° C. to 325° C. and a pressure in the range of from 0 bar to 140 bar. As used herein, the temperature refers to the temperature of the liquid phase in the reactor. In some embodiments, the temperature can be in the range of from 75° C. to 325° C., or from 100° C. to 325° C., or from 125° C. to 325° C., or from 150° C. to 320° C., or from 160° C. to 315° C., or from 170 to 310° C. In other embodiments, the temperature can be in the range of from 50° C. to 150° C., or from 65° C. to 140° C., or from 75° C. to 130° C. In still further embodiments, the temperature can be in the range of from 250° C. to 325° C., or from 260° C. to 320° C., or from 270° C. to 315° C., or from 275° C. to 310° C., or from 280° C. to 310° C. In some embodiments, the pressure can be in the range of from 5 bar to 130 bar, or from 15 bar to 120 bar, or from 20 bar to 120 bar. In other embodiments, the pressure can be in the range of from 1 bar to 5 bar, or 1 bar to 10 bar, or 1 bar to 20 bar. The pressure and temperature are chosen such that the reactor comprises a liquid component and at least a portion of the contents of the reactor in the gas phase. The individual pressure and temperature will be dependent upon the alcohol used for the contacting step a).

The alcohol can be an alcohol having in the range of from 1 to 12 carbon atoms. In some embodiments, the alcohol is an alkyl alcohol. Suitable alcohols can include, for example methanol, ethanol, propanol, butanol, pentanol, hexanol, heptanol, octanol, nonanol, decanol, undecanol, dodecanol, or isomers thereof. In some embodiments, the alcohol has in the range of from 1 to 6 carbon atoms, or in the range of from 1 to 4 carbon atoms, or in the range of from 1 to 2 carbon atoms. In some embodiments, the alcohol is methanol and the ester of FDCA is FDME.

In some embodiments, the FDCA can be fed to the reactor at a weight percentage in the range of from 1 to 50 percent of the feed, based on the total weight of the FDCA and the alcohol. Correspondingly, the alcohol can be present at a weight percentage of about 50 to 99 percent by weight, based on the total amount of FDCA and alcohol. In other embodiments, the FDCA can be present in the range of from 2 to 50 percent, or from 5 to 50 percent, or from 10 to 50 percent or from 15 to 50 percent 20 to 50 percent by weight, wherein all percentages by weight are based on the total amount of the FDCA and the alcohol.

In some embodiments, at least a portion of the alcohol can be replaced with an alcohol source. The alcohol source is a molecule which, in the presence of water and optionally an acid, forms an alcohol. In some embodiments, the alcohol source is an acetal, an orthoformate, an alkyl carbonate, a trialkyl borate, a cyclic ether comprising 3 or 4 atoms in the ring, or a combination thereof. Suitable acetals can include, for example, dialkyl acetals, wherein the alkyl portion of the acetal comprises in the range of from 1 to 12 carbon atoms. In some embodiments, the acetal can be 1,1-dimethoxyethane (acetaldehyde dimethyl acetal), 2,2 dimethoxypropane (acetone dimethyl acetal), 1,1-diethoxyethane (acetaldehyde diethyl acetal) or 2,2 diethoxypropane (acetone diethyl acetal). Suitable orthoformates can be, for example, trialkyl orthoformate wherein the alkyl group comprises in the range of from 1 to 12 carbon atoms. In some embodiments, the orthoester is trimethyl orthoformate or triethyl orthoformate. Suitable alkyl carbonates can be dialkyl carbonates wherein the alkyl portion comprises in the range of from 1 to 12 carbon atoms. In some embodiments, the dialkyl carbonate is dimethyl carbonate or diethyl carbonate. Suitable trialkyl borates can be, for example, trialkyl borates wherein the alkyl portion comprises in the range of from 1 to 12 carbon atoms. In some embodiments, the trialkyl borate is trimethyl borate or triethyl borate. A cyclic ether can also be used wherein the cyclic ether has 3 or 4 atoms in the ring. In some embodiments, the cyclic ether is ethylene oxide or oxetane.

In the above embodiments, the alcohol or the alcohol source can be used in the contacting step a). In further embodiments, combinations of the alcohol and the alcohol source can also be used. In some embodiments, the percentage by weight of the alcohol can be in the range of from 0.001 percent to 99.999 percent by weight, based on the total weight of the alcohol and the alcohol source. In other embodiments, the alcohol can be present at a percentage by weight in the range of from 1 to 99 percent or from 5 to 95 percent or from 10 to 90 percent or from 20 to 80 percent or from 30 to 70 percent or from 40 to 60 percent, wherein the percentages by weight are based on the total weight of the alcohol and the alcohol source.

The contacting step a) can optionally be performed in the presence of a catalyst. If present, the catalyst can be cobalt (II) acetate, iron (II) chloride, iron (III) chloride, iron (II) sulfate, iron (III) sulfate, iron (II) nitrate, iron (III) nitrate, iron (II) oxide, iron (III) oxide, iron (II) sulfide, iron (III) sulfide, iron (II) acetate, iron (III) acetate, magnesium (II) acetate, magnesium (II) hydroxide, manganese (II) acetate, phosphoric acid, sulfuric acid, zinc (II) acetate, zinc stearate, a solid acid catalyst, a zeolite solid catalyst or a combination thereof. The metal acetates, chlorides and hydroxides can be used as the hydrated salts. In some embodiments, the catalyst can be cobalt (II) acetate, iron (II) chloride, iron (III) chloride, magnesium (II) acetate, magnesium hydroxide, zinc (II) acetate or a hydrate thereof. In still further embodiments, the catalyst can be iron (II) chloride, iron (III) chloride or a combination thereof. In other embodiments, the catalyst can be cobalt acetate. In another embodiment, the catalyst can be sulfuric acid. Combinations of any of the above catalysts may also be useful. If present, a catalyst can be used at a rate of 0.1 to 5.0 percent by weight, based on the total weight of the FDCA, alcohol and optionally the alcohol source, and the catalyst. In other embodiments, the amount of catalyst present can be in the range of from 0.2 to 4.0 or from 0.5 to 3.0 or from 0.75 to 2.0 or from 1.0 to 1.5 percent by weight, wherein the percentages by weight are based on the total amount of FDCA, methanol and the catalyst.

The catalyst can also be a solid acid catalyst having the thermal stability required to survive reaction conditions. The solid acid catalyst may be supported on at least one catalyst support. Examples of suitable solid acids include without limitation the following categories: 1) heterogeneous heteropolyacids (HPAs) and their salts, 2) natural or synthetic minerals (including both clays and zeolites), such as those containing alumina and/or silica, 3) cation exchange resins, 4) metal oxides, 5) mixed metal oxides, 6) metal salts such as metal sulfides, metal sulfates, metal sulfonates, metal nitrates, metal phosphates, metal phosphonates, metal molybdates, metal tungstates, metal borates or combinations thereof. The metal components of categories 4 to 6 may be selected from elements from Groups 1 through 12 of the Periodic Table of the Elements, as well as aluminum, chromium, tin, titanium, and zirconium. Examples include, without limitation, sulfated zirconia and sulfated titania.

Suitable HPAs include compounds of the general formula XₐM_{b}O_{c}^{q-}, where X is a heteroatom such as phosphorus, silicon, boron, aluminum, germanium, titanium, zirconium, cerium, cobalt or chromium, M is at least one transition metal such as tungsten, molybdenum, niobium, vanadium, or tantalum, and q, a, b, and c are individually selected whole numbers or fractions thereof. Nonlimiting examples of salts of HPAs include, for example, lithium, sodium, potassium, cesium, magnesium, barium, copper, gold and gallium, and ammonium salts. Examples of HPAs suitable for the disclosed process include, but are not limited to, tungstosilicic acid (H₄[SiW₁₂O₄₀].xH₂O), tungstophosphoric acid (H₃[PW₁₂O₄₀].xH₂O), molybdophosphoric acid (H₃[PMo₁₂O₄₀].xH₂O), molybdosilicic acid (H₄[SiMo₁₂O₄₀].xH2O), vanadotungstosilicic acid (H₄₊ₙ[SiVₙW₁₂₋ₙO₄₀].xH₂O), vanadotungstophosphoric acid (H₃₊ₙ[PVₙW₁₂₋ₙO₄₀].xH₂O), vanadomolybdophosphoric acid (H₃₊ₙ[PVₙMo₁₂₋ₙO₄₀].xH₂O), vanadomolybdosilicic acid (H₄₊ₙ[SiVₙMo₁₂₋ₙO₄₀].xH₂O), molybdotungstosilicic acid (H₄[SiMoₙW_{12631 n}O₄₀].xH₂O), molybdotungstophosphoric acid (H₃[PMoₙW_{12631 n}O₄₀].xH₂O), wherein n in the formulas is an integer from 1 to 11 and x is an integer of 1 or more.

Natural clay minerals are well known in the art and include, without limitation, kaolinite, bentonite, attapulgite, and montmorillonite.

In an embodiment, the solid acid catalyst is a cation exchange resin that is a sulfonic acid functionalized polymer. Suitable cation exchange resins include, but are not limited to the following: styrene divinylbenzene copolymer-based strong cation exchange resins such as AMBERLYST™ and DOWEX® available from Dow Chemicals (Midland, Mich.) (for example, DOWEX® Monosphere M-31, AMBERLYST™ 15, AMBERLITE™ 120); CG resins available from Resintech, Inc. (West Berlin, N.J.); Lewatit resins such as MONOPLUST™ S 100H available from Sybron Chemicals Inc. (Birmingham, N.J.); fluorinated sulfonic acid polymers (these acids are partially or totally fluorinated hydrocarbon polymers containing pendant sulfonic acid groups, which may be partially or totally converted to the salt form) such as NAFION® perfluorinated sulfonic acid polymer, NAFION® Super Acid Catalyst (a bead-form strongly acidic resin which is a copolymer of tetrafluoroethylene and perfluoro-3,6-dioxa-4-methyl-7-octene sulfonyl fluoride, converted to either the proton (H⁺), or the metal salt form) available from DuPont Company (Wilmington, Del.).

In an embodiment, the solid acid catalyst is a supported acid catalyst. The support for the solid acid catalyst can be any solid substance that is inert under the reaction conditions including, but not limited to, oxides such as silica, alumina, titania, sulfated titania, and compounds thereof and combinations thereof; barium sulfate; calcium carbonate; zirconia; carbons, particularly acid washed carbon; and combinations thereof. Acid washed carbon is a carbon that has been washed with an acid, such as nitric acid, sulfuric acid or acetic acid, to remove impurities. The support can be in the form of powder, granules, pellets, or the like. The supported acid catalyst can be prepared by depositing the acid catalyst on the support by any number of methods well known to those skilled in the art of catalysis, such as spraying, soaking or physical mixing, followed by drying, calcination, and if necessary, activation through methods such as reduction or oxidation. The loading of the at least one acid catalyst on the at least one support is in the range of 0.1-20 weight percent based on the combined weights of the at least one acid catalyst and the at least one support. Certain acid catalysts perform better at low loadings such as 0.1-5%, whereas other acid catalysts are more likely to be useful at higher loadings such as 10-20%. In an embodiment, the acid catalyst is an unsupported catalyst having 100% acid catalyst with no support such as, pure zeolites and acidic ion exchange resins.

Examples of supported solid acid catalysts include, but are not limited to, phosphoric acid on silica, NAFION®, a sulfonated perfluorinated polymer, HPAs on silica, sulfated zirconia, and sulfated titania. In the case of NAFION® on silica, a loading of 12.5% is typical of commercial examples.

In another embodiment, the solid acid catalyst comprises a sulfonated divinylbenzene/styrene copolymer, such as AMBERLYST™ 70.

In one embodiment, the solid acid catalyst comprises a sulfonated perfluorinated polymer, such as NAFION® supported on silica (SiO₂).

In one embodiment, the solid acid catalyst comprises natural or synthetic minerals (including both clays and zeolites), such as those containing alumina and/or silica.

Zeolites suitable for use herein can be generally represented by the following formula M_{2/n}O.AI₂O₃.xSiO₂.yH₂O wherein M is a cation of valence n, x is greater than or equal to about 2, and y is a number determined by the porosity and the hydration state of the zeolite, generally from about 2 to about 8. In naturally occurring zeolites, M is principally represented by Na, Ca, K, Mg and Ba in proportions usually reflecting their approximate geochemical abundance. The cations M are loosely bound to the structure and can frequently be completely or partially replaced with other cations by conventional ion exchange.

The zeolite framework structure has corner-linked tetrahedra with Al or Si atoms at centers of the tetrahedra and oxygen atoms at the corners. Such tetrahedra are combined in a well-defined repeating structure comprising various combinations of 4-, 6-, 8-, 10-, and 12-membered rings. The resulting framework structure is a pore network of regular channels and cages that is useful for separation. Pore dimensions are determined by the geometry of the aluminosilicate tetrahedra forming the zeolite channels or cages, with nominal openings of about 0.26 nm for 6-member rings, about 0.40 nm for 8-member rings, about 0.55 nm for 10-member rings, and about 0.74 nm for 12-member rings (these numbers assume the ionic radii for oxygen). Zeolites with the largest pores, being 8-member rings, 10-member rings, and 12-member rings, are frequently considered small, medium and large pore zeolites, respectively.

In a zeolite, the term “silicon to aluminum ratio” or, equivalently, “Si/AI ratio” means the ratio of silicon atoms to aluminum atoms. Pore dimensions are critical to the performance of these materials in catalytic and separation applications, since this characteristic determines whether molecules of certain size can enter and exit the zeolite framework.

In practice, it has been observed that very slight decreases in ring dimensions can effectively hinder or block movement of particular molecular species through the zeolite structure. The effective pore dimensions that control access to the interior of the zeolites are determined not only by the geometric dimensions of the tetrahedra forming the pore opening, but also by the presence or absence of ions in or near the pore. For example, in the case of zeolite type A, access can be restricted by monovalent ions, such as Na⁺ or K⁺, which are situated in or near 8-member ring openings as well as 6-member ring openings. Access can be enhanced by divalent ions, such as Ca²⁺, which are situated only in or near 6-member ring openings. Thus, the potassium and sodium salts of zeolite A exhibit effective pore openings of about 0.3 nm and about 0.4 nm respectively, whereas the calcium salt of zeolite A has an effective pore opening of about 0.5 nm.

The presence or absence of ions in or near the pores, channels and/or cages can also significantly modify the accessible pore volume of the zeolite for sorbing materials. Representative examples of zeolites are (i) small pore zeolites such as NaA (LTA), CaA (LTA), Erionite (ERI), Rho (RHO), ZK-5 (KFI) and chabazite (CHA); (ii) medium pore zeolites such as ZSM-5 ZSM-11 (MEL), ZSM -22 (TON), and ZSM-48 (*MRE); and (iii) large pore zeolites such as zeolite beta (BEA), faujasite (FAU), mordenite (MOR), zeolite L (LTL), NaX (FAU), NaY (FAU), DA-Y (FAU) and CaY (FAU). The letters in parentheses give the framework structure type of the zeolite.

Zeolites suitable for use herein include medium or large pore, acidic, hydrophobic zeolites, including without limitation ZSM-5, faujasites, beta, mordenite zeolites or mixtures thereof, having a high silicon to aluminum ratio, such as in the range of 5:1 to 400:1 or 5:1 to 200:1. Medium pore zeolites have a framework structure consisting of 10-membered rings with a pore size of about 0.5-0.6 nm. Large pore zeolites have a framework structure consisting of 12-membered rings with a pore size of about 0.65 to about 0.75 nm. Hydrophobic zeolites generally have Si/AI ratios greater than or equal to about 5, and the hydrophobicity generally increases with increasing Si/AI ratios. Other suitable zeolites include without limitation acidic large pore zeolites such as H-Y with Si/AI in the range of about 2.25 to 5.

The contacting step a) can produce a mixture comprising the ester of FDCA, the alcohol and water. The mixture can also comprise the monoester of FDCA, and an alkyl ester of 5-formylfuran-2-carboxylic acid.

In some embodiments, the alcohol is methanol and the mixture comprises methanol, the dimethyl ester of FDCA, the monomethyl ester of FDCA, methyl 5-formylfuran-2-carboxylate and water. If an alcohol source is used, then the mixture can also comprise one or more of the by-products from the hydrolysis of the alcohol source. For example, in the presence of water, trimethyl orthoformate is known to form methanol and methyl formate. Other hydrolysis products of the disclosed alcohol sources are well-known in the art and can be present in the mother liquor.

The process further comprises a step b) removing at least a portion of a gas phase from the mixture. This means that the gas phase is removed from the reaction vessel. In the case of successive tank reactors or a series of continuously stirred tank reactors, gas phase removal can be accomplished via a gas phase outlet in each reactor. In the case of a reaction column, removal of the gas phase is accomplished by at least a portion of the gas phase exiting the first stage prior to the bulk liquid of the first stage moving to the second stage. At each stage, the gas phase exits prior to the liquid phase as the reaction proceeds to each successive stage. The gas phase of the reactor comprises alcohol, water and the ester of FDCA. Removing at least a portion of the gas phase, can help to remove water from the reactor which can help drive the equilibrium esterification reaction towards the ester. As each stage of the n-stage reactor system comprises both a liquid phase and a gas phase, as long as at least some of the gas phase is removed through, for example, a vapor outlet, then at least a portion of the contents of the reactor can then be moved to the next sequential stage of the n-stage reactor system.

The process then comprises a step c), allowing at least a portion of the mixture to move to the next sequential reactor in the series. If the reactor was the first reactor of the n-stage reactor system, then at least a portion of the mixture is transferred to the second reactor of the n-stage reactor system. If the reactor was the second stage of the n-stage reactor system, and the n-stage reactor system comprises at least three stages, then at least a portion of the mixture is moved to the third reactor of the n-stage reactor system. Moving the mixture can be accomplished via gravity, the mixture can be pumped or a combination of gravity and pumping can move at least a portion of the mixture to the next stage of the n-stage reactor system.

The process further comprises a step d) heating the mixture from step c) to a temperature in the range of from 150° C. to 325° C. and a pressure in the range of from 0 bar to 140 bar. The temperature and pressure conditions of each stage of the n-stage reactor system can be identical in each step or the conditions can be chosen independently of one another. The individual temperature and pressure conditions can be chosen from the various temperature and pressure parameters disclosed for the contacting step a) and are selected so that at least a portion of the contents of the reactor are in the liquid phase, and at least a portion are in a gas phase. Each successive step d) of heating the mixture, in general, increases the amount of ester of FDCA, that is, the diester of FDCA, when compared to the previous heating step. In this way, as the mixture progresses through each stage of the n-stage reactor system, higher and higher yields of the desired diester product of FDCA can be realized.

Step e) of the process comprises removing at least a portion of the gas phase. The removal step e) can be carried out using the vapor removal outlet of the individual stage reactor.

Step f) of the process is to repeat step c), d) and e) until the last stage of the n-stage reactor system is reached. In some embodiments, the n-stage reactor system can comprise 2, 3, 4, 5, 6, 7, 8, 9 or 10 stages. If the n-stage reactor system comprises 2 stages, then only steps a), b), c), d) and e) are completed prior to moving to step g).

The process further comprises step g), separating the ester of FDCA from the mixture. The separation step g) can be a distillation step, a crystallization step, or a combination thereof. In some embodiments, the separation step g) is a distillation step, wherein the distillation is performed at low pressure, for example, in the range of from less than 1 bar to 0.0001 bar. In other embodiments, the pressure can be in the range of from 0.75 bar to 0.001 bar or from 0.5 bar to 0.01 bar. The separation step g) can also be a crystallization step wherein the contents of the reactor are cooled to crystallize the ester of FDCA. The cooling step can be accomplished in the last reactor, in a separate cooling vessel or in a series of cooling vessels. The crystallized ester of FDCA can be separated from the liquid component, for example, the alcohol and the water, by filtration or by centrifugation. The solids obtained from the separation can further be recrystallized from any of the known recrystallization solvents, for example, methanol, ethanol, propanol or butanol. The solids can be distilled or they can be sublimed to produce a relatively pure ester of FDCA. In some embodiments, the separation step g) can be accomplished by the distillation step followed by the recrystallization step or by both the recrystallization step followed by the distillation step.

The processes disclosed herein can result in an ester of FDCA containing less than 50 parts per million (ppm) of any one of the impurities. For example, the ester of FDCA from step g) can contain less than 50 ppm of the alkyl ester of 5-formylfuran-2-carboxylic acid, less than 50 ppm of the monoalkyl ester of 2,5-furan dicarboxylic acid and/or less than 50 ppm FDCA. In other embodiments, the ester of FDCA from step g) can contain less than 25 ppm of the alkyl ester of 5-formylfuran-2-carboxylic acid, less than 25 ppm of the monoalkyl ester of 2,5-furan dicarboxylic acid and/or less than 25 ppm FDCA. In still further embodiments, the ester of FDCA from step g) can contain less than 10 ppm of the alkyl ester of 5-formylfuran-2-carboxylic acid, less than 10 ppm the monoalkyl ester of 2,5-furan dicarboxylic acid and/or less than 10 ppm FDCA.

Non-limiting examples of the processes disclosed herein include:
1. A process comprising:
   a) contacting FDCA with excess alcohol and, optionally, a catalyst in a first stage of a n-stage reactor at a temperature in the range of from 50° C. to 325° C. and a pressure in the range of from 0 bar to 140 bar to form a mixture comprising an ester of FDCA, the alcohol and water;
   b) removing at least a portion of a gas phase from the first stage of the n-stage reactor;
   c) allowing at least a portion of the mixture to move to the next sequential stage of the n-stage reactor system;
   d) heating the mixture of step c) to a temperature in the range of from 50° C. to 325° C. and a pressure in the range of from 0 bar to 140 bar;
   e) removing at least a portion of a gas phase from the mixture of step d);
   f) repeating steps c), d) and e) until the last stage of the n-stage reactor system is reached; and
   g) separating the ester of FDCA from the mixture;
   wherein n is 2 to 10.
2. The process of embodiment 1 wherein the ester of FDCA of step g) is further purified by crystallization, distillation or a combination thereof.
3. The process of any one of embodiments 1 or 2 wherein the gas phases removed in steps b) and e) comprise alcohol, water, the ester of FDCA or a combination thereof.
4. The process of any one of embodiments 1, 2 or 3 wherein the n-stage reactor system comprises one or more of a continuously stirred tank reactor, a plug flow reactor, a reaction column, a Scheibel column, a tank reactor or a combination thereof.
5. The process of embodiment 4 wherein the n-stage reactor system comprises continuously stirred tank reactors connected in series.
6. The process of any one of embodiments 1, 2, 3, 4 or 5 wherein the alcohol is methanol and the ester of FDCA is FDME.
7. The process of any one of embodiments 1, 2, 3, 4, 5 or 6 wherein the catalyst is cobalt (II) acetate, iron (II) chloride, iron (III) chloride, iron (II) sulfate, iron (III) sulfate, iron (II) nitrate, iron (III) nitrate, iron (II) oxide, iron (III) oxide, iron (II) sulfide, iron (III) sulfide, iron (II) acetate, iron (III) acetate, magnesium (II) acetate, magnesium (II) hydroxide, manganese (II) acetate, phosphoric acid, sulfuric acid, zinc (II) acetate, zinc stearate, a solid acid catalyst, a zeolite solid catalyst or a combination thereof.
8. The process of any one of embodiments 1, 2, 3, 4, 5, 6 or 7 wherein at least a portion of the alcohol is replaced with an alcohol source.
9. The process of any one of embodiments 1, 2, 3, 4, 5, 6, 7, or 8 wherein the alcohol source is an acetal, an orthoformate, an alkyl carbonate, a trialkyl borate, a cyclic ether comprising 3 or 4 atoms in the ring, or a combination thereof.

### EXAMPLES

The disclosed process has been modeled using an Aspen flowsheet, ASPEN PLUS® v8.4 (available from Aspen Technology, Inc., Bedford, Mass.). The examples below illustrate the effect of reactor staging with and without water removal. The reaction rates were predicted from a kinetic expression modeling the first and second esterification reactions as reversible reactions. For all cases, the feed to the system is held constant at 20 percent by weight FDCA and 80 percent by weight methanol. The reactor operating temperature and pressures are kept constant at 220° C. and 70 bar (1000 psig). The overall residence time of the three-stage reactor system was 30 minutes.

Comparative Case 1—a single continuously stirred tank reactor.

Comparative Case 2-3 equal volume continuously stirred tank reactors in series.

### Example 1-3 equal volume continuously stirred tank reactors with interstage water removal

The model predictions in TABLE 1 show the advantage of performing the reaction in an n-stage reactor system.

TABLE 1

| | Comparative | Comparative | |
|---|---|---|---|
| | Case 1 | Case 2 | Example 1 |
| FDCA | 24% | 13% | 14% |
| FDME | 39% | 46% | 54% |
| FDMME | 26% | 28% | 30% |
| Water | 10% | 12% | 2% |

What is claimed is:

1. A process comprising:
a) contacting 2,5-furan dicarboxylic acid with excess alcohol and, optionally, a catalyst in a first stage of a n-stage reactor at a temperature in the range of from 50° C. to 325° C. and a pressure in the range of from 0 bar to 140 bar to form a mixture comprising an ester of 2,5-furan dicarboxylic acid, the alcohol and water;
b) removing at least a portion of a gas phase from the first stage of the n-stage reactor;
c) allowing at least a portion of the mixture to move to the next sequential stage of the n-stage reactor system;
d) heating the mixture of step c) to a temperature in the range of from 50° C. to 325° C. and a pressure in the range of from 0 bar to 140 bar;
e) removing at least a portion of a gas phase from the mixture of step d);
f) repeating steps c), d) and e) until the last stage of the n-stage reactor system is reached; and
g) separating the ester of 2,5-furan dicarboxylic acid from the mixture;
wherein n is 2 to 10.

2. The process of claim 1 wherein the ester of 2,5-furan dicarboxylic acid of step g) is further purified by crystallization, distillation, or a combination thereof.

3. The process of claim 1 wherein the gas phases removed in steps b) and e) comprise alcohol, water, the ester of 2,5-furan dicarboxylic acid, or a combination thereof.

4. The process of claim 1 wherein the n-stage reactor system comprises one or more of a continuously stirred tank reactor, a plug flow reactor, a reaction column, a Scheibel column, a tank reactor, or a combination thereof.

5. The process of claim 4 wherein the n-stage reactor system comprises continuously stirred tank reactors connected in series.

6. The process of claim 1 wherein the alcohol is methanol and the ester of 2,5-furan dicarboxylic acid is the dimethyl ester of 2,5-furan dicarboxylic acid.

7. The process of claim 1 wherein the catalyst is cobalt (II) acetate, iron (II) chloride, iron (Ill) chloride, iron (II) sulfate, iron (III) sulfate, iron (II) nitrate, iron (III) nitrate, iron (II) oxide, iron (III) oxide, iron (II) sulfide, iron (III) sulfide, iron (II) acetate, iron (III) acetate, magnesium (II) acetate, magnesium (II) hydroxide, manganese (II) acetate, phosphoric acid, sulfuric acid, zinc (II) acetate, zinc stearate, a solid acid catalyst, a zeolite solid catalyst or a combination thereof.

8. The process of claim 1, wherein at least a portion of the alcohol is replaced with an alcohol source.

9. The process of claim 8, wherein the alcohol source is an acetal, an orthoformate, an alkyl carbonate, a trialkyl borate, a cyclic ether comprising 3 or 4 atoms in the ring, or a combination thereof.
